(19) United States
(12) Patent Application Publication
(10) Pub. No.: US 2026/0191431 A1
(43) Pub. Date: Jul. 9, 2026

(54) NOISE REJECTION METHODS AND APPARATUS FOR SPARSELY SAMPLED ANALYTE SENSOR DATA

(71) Applicant: ABBOTT DIABETES CARE INC., Alameda, CA (US)

(72) Inventor: Erwin Satrya Budiman, Fremont, CA (US)

(73) Assignee: ABBOTT DIABETES CARE INC., Alameda, CA (US)

(21) Appl. No.: 19/264,103

(22) Filed: Jul. 9, 2025

Related U.S. Application Data
(63) Continuation of application No. 16/595,086, filed on Oct. 7, 2019, now Pat. No. 12,383,164.

(51) Int. Cl. A61B 5/145; A61B 5/0205; G01N 33/00

(52) U.S. Cl. CPC A61B 5/145; A61B 5/14532; A61B 5/0205; G01N 33/00

(57) ABSTRACT

Systems, methods and apparatus are provided for rejecting noise from sparsely sampled analyte sensor data. Embodiments of the present disclosure include receiving a raw set of sensor data from an on-body device including an in vivo analyte sensor, determining an interpolation-based estimate of an analyte level over time based on the raw set of sensor data, determining an extrapolation-based estimate of the analyte level over time based on the raw set of sensor data, determining a combined estimate of the analyte level over time based on the interpolation-based estimate and the extrapolation-based estimate, and displaying a representation of the combined estimate of the analyte level over time on an output device. Numerous additional aspects are disclosed.

### RELATED APPLICATION

The present application claims priority under 35 U.S.C. § 119(e) to U.S. Provisional Application No. 61/794,549 filed Mar. 15, 2013, entitled “Noise Rejection Methods and Apparatus For Sparsely Sampled Analyte Sensor Data,” the disclosure of which is incorporated herein by reference for all purposes.

### BACKGROUND

The detection of the concentration level of glucose or other analytes in certain individuals may be vitally important to their health. For example, the monitoring of glucose levels is particularly important to individuals with diabetes or pre-diabetes. People with diabetes may need to monitor their glucose levels to determine when medication (e.g., insulin) is needed to reduce their glucose levels or when additional glucose is needed.

Devices have been developed for automated in vivo monitoring of analyte time series characteristics, such as glucose levels, in bodily fluids such as in the blood stream or in interstitial fluid. Some of these analyte level measuring devices are configured so that at least a portion of a sensor of an on-body device is positioned below a skin surface of a user, e.g., in a blood vessel or in the subcutaneous tissue of a user. As used herein, the term analyte monitoring system is used to refer to any type of in vivo monitoring system that uses a sensor disposed with at least a subcutaneous portion to measure and store sensor data representative of analyte concentration levels automatically over time. Analyte monitoring systems include both (1) systems such as continuous glucose monitors (CGMs) which transmit sensor data continuously or at regular time intervals (e.g., once per minute) to a processor/display unit and (2) systems that transfer stored sensor data in one or more batches in response to data request from a processor/display unit (e.g., based on an activation action and/or proximity using, for example, a near field communications protocol).

Some analyte monitoring systems may store samples relatively infrequently. For example, the sensor data may only include measurements or samples taken once every ten or fifteen minutes. In some cases, such sparsely sampled analyte sensor data may not accurately reflect the analyte concentration levels, particularly if signal noise is present. Thus, what are needed are systems, methods and apparatus that can reliably represent the analyte concentration level even of sparsely sampled data is used.

### SUMMARY

As mentioned above, accurate monitoring of analyte levels can be important to a person's health. To insure that sensor data does accurately reflect analyte concentration, embodiments of the present disclosure provide systems, methods, and apparatus for rejecting noise from sparsely sampled analyte sensor data that does not alter or distort true sensor data excursions. Conventional noise filtering from sparsely sampled sensor data can result in undesirable side effects such as over-filtering, particularly where an actual rapid change (e.g., a relatively fast change compared to the sample rate) in analyte concentration (i.e., a fast true sensor data excursion) occurs. In effect, conventional analyte sensor data filtering methods may not reliably distinguish between noise that should be rejected and rapid changes in analyte concentration that should be preserved. As a result, the analyte sensor can appear less responsive, and, in addition, can lag as compared to reference analyte measurements. The present disclosure provides novel noise rejection methods that take advantage of the similarities and differences of interpolation-based and extrapolation-based estimation methods to filter noise without attenuating fast true sensor data excursions.

In some embodiments, the present disclosure provides systems, methods and apparatus for rejecting noise from sparsely sampled analyte sensor data. The invention includes receiving a raw set of sensor data from an on-body device including an in vivo analyte sensor, determining an interpolation-based estimate of an analyte level over time based on the raw set of sensor data, determining an extrapolation-based estimate of the analyte level over time based on the raw set of sensor data, determining a combined estimate of the analyte level over time based on the interpolation-based estimate and the extrapolation-based estimate, and displaying a representation of the combined estimate of the analyte level over time on an output device.

The invention also includes a computer system and a computer program product for rejecting noise in sparsely sampled analyte monitoring system sensor data. Numerous other aspects and embodiments are provided. Other features and aspects of the present disclosure will become more fully apparent from the following detailed description, the appended claims, and the accompanying drawings.

### BRIEF DESCRIPTION OF THE DRAWINGS

The accompanying drawings, which are incorporated herein, form part of the specification. Together with this written description, the drawings further serve to explain the principles of, and to enable a person skilled in the relevant arts, to make and use the present disclosure.

FIG. 1 depicts an example graph illustrating a Least Squares fit of a straight line to estimate a sensor data value in accordance with some embodiments of the present disclosure.

FIG. 2 depicts an example graph illustrating the smoothing effect of the Least Squares fit based calculation in accordance with some embodiments of the present disclosure.

FIG. 3 depicts an example graph illustrating the Least Squares fit based calculation applied outside the measurement window in accordance with some embodiments of the present disclosure.

FIG. 4 depicts an example graph illustrating the combination of an interpolation-based calculation and an extrapolation-based calculation in accordance with some embodiments of the present disclosure.

FIG. 5 depicts an example graph illustrating the effectiveness of applying methods of the present disclosure to noisy sensor data in accordance with some embodiments of the present disclosure.

FIG. 6 is a flow chart depicting an example method of noise rejection for sparsely sampled analyte sensor data in accordance with some embodiments of the present disclosure.

### DETAILED DESCRIPTION

Before the embodiments of the present disclosure are described, it is to be understood that this invention is not limited to the particular embodiments described, as such may, of course, vary. It is also to be understood that the terminology used herein is for the purpose of describing particular embodiments only, and is not intended to be limiting, since the scope of the embodiments of the invention will be limited only by the appended claims.

The present disclosure provides systems, methods, and apparatus to reject noise from sparsely sampled analyte sensor data that does not alter or distort true sensor data excursions. As used herein, the term “sparsely sampled” is intended to mean a sample rate that is low enough such that first phase responses to meal and/or insulin may be difficult to discern in real-time. For example, based on average human physiology, a sample rate of once every ten minutes or slower is a sparsely sampled rate. The invention can be applied to sensor data from an analyte monitoring system, such as, for example, any type of in vivo monitoring system that uses a sensor disposed with at least a subcutaneous portion to measure and store sensor data representative of analyte concentration levels automatically over time. Analyte monitoring systems may include CGMs which are programmed to transmit sensor data according to a predetermined transmission schedule, continuously, or at regular time intervals to a processor/display unit and systems that transfer stored sensor data in one or more batches in response to a request from a processor/display unit, i.e., not according to a predetermined transmission schedule. Without requiring a patient to provide blood samples for in vitro reference glucose readings, the present disclosure is operable to reject noise from sparsely sampled data from an in vivo analyte sensor.

According to some embodiments of the present disclosure, a dataset representative of a patient's monitored analyte concentration level (herein referred to as “sensor data”) over time is received from an on-body device that includes sensor electronics operatively coupled to an analyte sensor that is in fluid contact with interstitial fluid. In some embodiments, the sensor data may represent a collection of data received from the on-body device at several different times during a wear period of the on-body device. In some other embodiments, the sensor data may represent data collected and stored over an entire wear period of the on-body device and only received from the on-body device at the end of the wear period or at the end of the useful life of the on-body device. In other words, the sensor data can be transmitted continuously, on a regular schedule, in multiple batches over time, in batches on demand, or in a single batch.

Embodiments of the present disclosure may be applied to any analyte concentration level determination system that may exhibit or at least be suspected of exhibiting, or that may be susceptible to noise in the sensor data. Embodiments of the invention are described primarily with respect to continuous glucose monitoring devices and systems but the present disclosure may be applied to other analytes and analyte characteristics, as well as data from measurement systems that transmit sensor data from a sensor unit to another unit such as a processing or display unit in response to a request from the other unit. For example, other analytes that may be monitored include, but are not limited to, acetyl choline, amylase, bilirubin, cholesterol, chorionic gonadotropin, creatine kinase (e.g., CK-MB), creatine, DNA, fructosamine, glutamine, growth hormones, hormones, ketones, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In the embodiments that monitor more than one analyte, the analytes may be monitored at the same or different times. The present disclosure also provides numerous additional embodiments.

Embodiments of the present disclosure may include a programmed computer system adapted to receive and store data from an analyte monitoring system. The computer system may include one or more processors for executing instructions or programs that implement the methods described herein. The computer system may include memory and persistent storage devices to store and manipulate the instructions and sensor data received from the analyte monitoring system. The computer system may also include communications facilities (e.g., wireless and/or wired) to enable transfer of the sensor data from the analyte monitoring system to the computer. The computer system may include a display and/or output devices for identifying dropouts in the sensor data to a user. The computer system may include input devices and various other components (e.g., power supply, operating system, clock, etc.) that are typically found in a conventional computer system. In some embodiments, the computer system may be integral to the analyte monitoring system. For example, the computer system may be embodied as a handheld or portable receiver unit within the analyte monitoring system.

The various methods described herein for performing one or more processes also described herein may be embodied as computer programs (e.g., computer executable instructions and data structures) developed using an object oriented programming language that allows the modeling of complex systems with modular objects to create abstractions that are representative of real world, physical objects and their interrelationships. However, any practicable programming language and/or techniques may be used. The software for performing the inventive processes, which may be stored in a memory or storage device of the computer system described herein, may be developed by a person of ordinary skill in the art based upon the present disclosure and may include one or more computer program products.

The computer program products may be stored on a computer readable medium such as a server memory, a computer network, the Internet, and/or a computer storage device. Note that in some cases the methods embodied as software may be described herein with respect to a particular order of operation or execution. However, it will be understood by one of ordinary skill that any practicable order of operation or execution is possible and such variations are contemplated by this specification of the present disclosure.

Rejecting noise can be essential in generating an accurate representation of an analyte concentration level using an analyte monitoring system. In some analyte monitoring systems, for example, the sensor data can include a window of sparsely sampled data long enough to cover a significant portion of a day, e.g., a 6 to 12 hour window with datapoints every 10 to 20 minutes. In addition to noise, some of the data points may not be available due to data quality issues. A reliable analyte measurement system according to the present disclosure can reject noise and recover missing data using the remaining sparsely sampled data.

Conventional filtering methods can apply a relatively simple approach that is robust to intermittent signal loss and noise. This method includes fitting one or more parameters of a pre-determined polynomial structure over a window of sensor data using the Least-Squares Error (LS) fit method. An analytical solution can be derived for each of the parameters, as long as there is sufficiency of excitation and the number of parameters identified remain small (e.g. up to 3 parameters). This means that polynomials with up to three degrees of freedom (e.g., linear (a straight line with 0 intercept); affine (straight line with general intercept); or parabolic) can be considered. For numerical robustness with respect to noise, affine functions are considered. Up to two parameters are estimated, namely the slope and intercept.

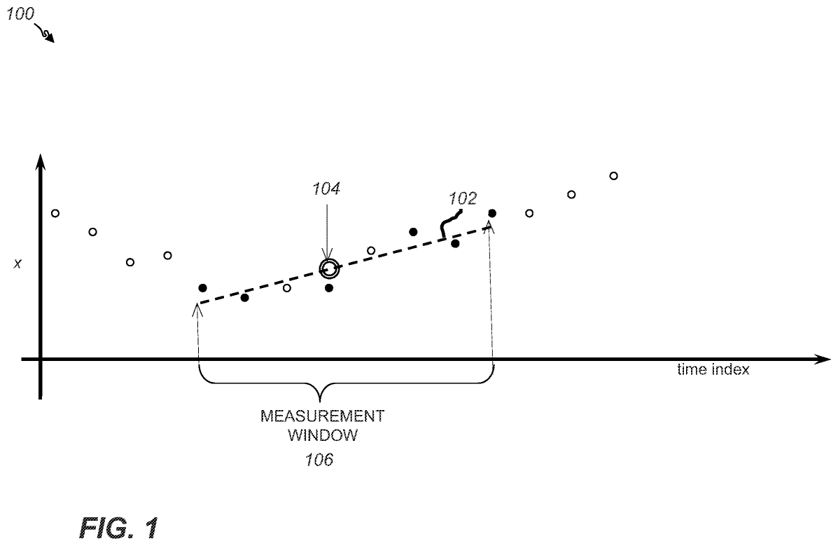

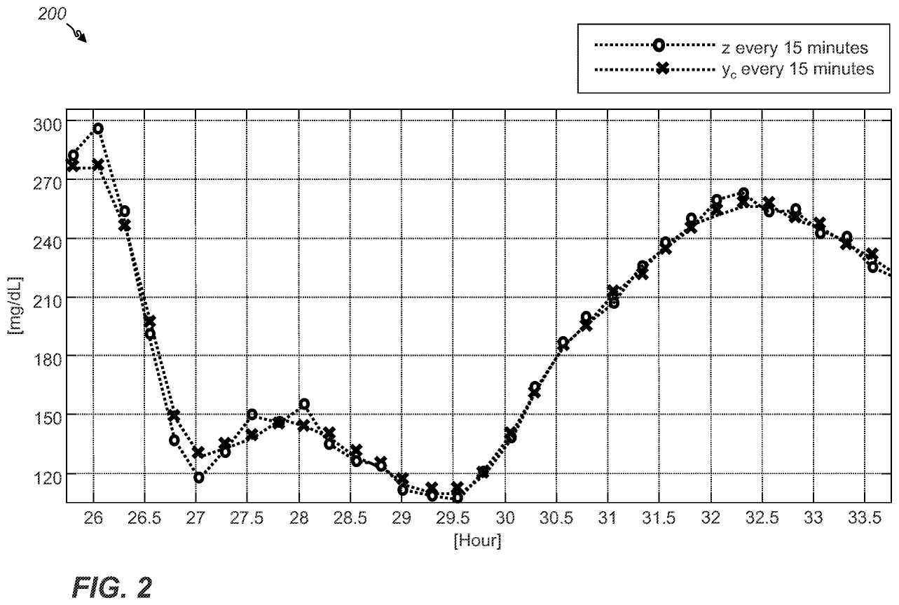

FIG. 1 is a graph 100 of sensor data values plotted over time. The graph 100 illustrates an example of a LS fit of a straight line 102 to estimate a value at a time of interest 104 and the rate of change (i.e. the slope of the LS fit), whether or not the source data at time of interest 104 is available. In the example shown in FIG. 1, the time of interest 104 is inside the measurement window 106. The filled solid circles represent available and valid data points within the measurement window 106. As will be shown in more detail with respect to FIG. 2, the LS fit method allows recovery of missing data based on neighboring raw sensor data. However, as will also be illustrated below, obtaining an estimate outside the window 106, whose instance is relatively distant from the center of the measurement window 106 and large relative to the size of the measurement window 106, can exaggerate the negative effects of extrapolation.

LS fit of a straight line of data in a measurement window can be used to achieve robust signal recovery and noise rejection. For example, suppose an LS fit of a straight line is determined using three data points spaced fifteen minutes apart and the LS fit estimate at the center data point is used as the output. FIG. 2 depicts a graph 200 of an example of a raw sensor data set plot z stored at fifteen minute intervals. In this example, curve y_{c} is the resulting LS fit estimate using the method described above with respect to FIG. 1, based on three z neighboring values and estimating the center value. In general, curve y_{c} is a smoother representation of the sensor data values compared to plot z. However, the values around fast transitions (e.g., around the peak at 26 Hr. and valley at 27 Hr.) are severely attenuated, which is evident from the reduced dynamic range (or roughly peak-to-peak distance) of the LS fit result compared to plot z.

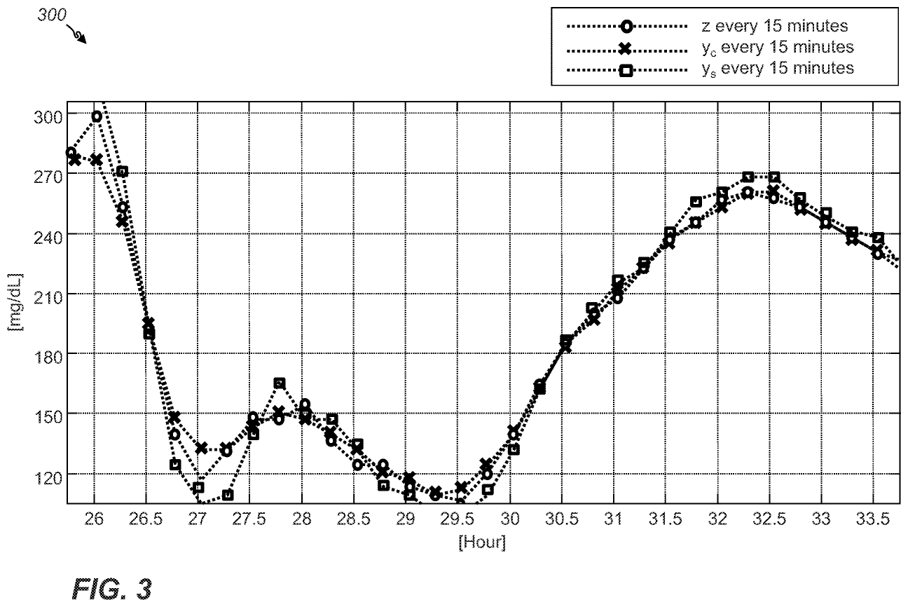

When the LS fit is used to estimate a value at the edge of the window or slightly outside of the window, the attenuation rounding effect is replaced by noise amplification associated with extrapolation. However, when results of two extrapolations (or near extrapolation in the case where the estimate lies on the edge of the window) are combined such that the estimate lies on the same sample instance, and one window uses past data while the other uses future data, the result is a reasonably smooth signal with exaggerated sharp apexes. This result is shown as curve yₛ in the graph 300 of FIG. 3, which is generally smoother than plot z, but errs on the opposite side of plot z compared to curve y_{c}.

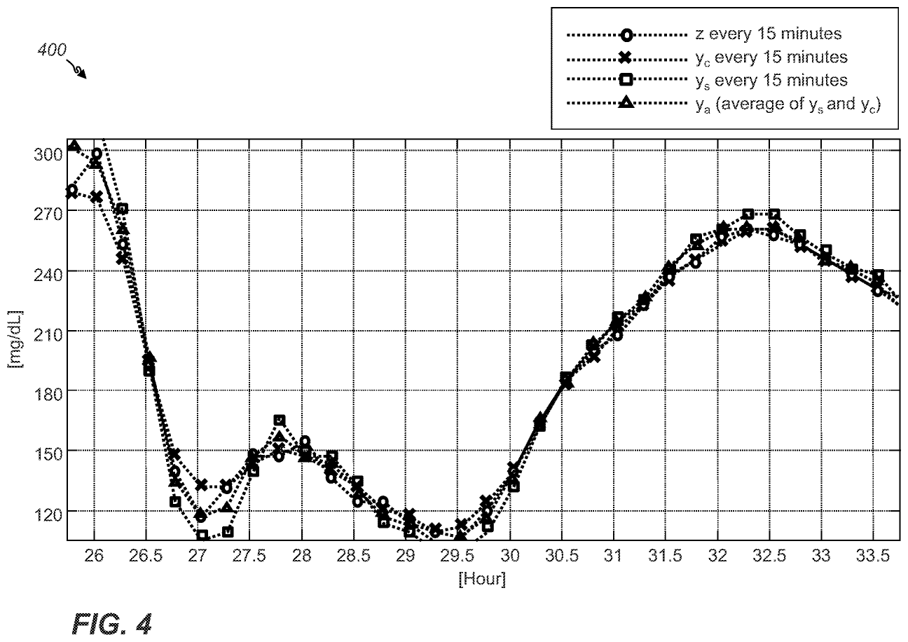

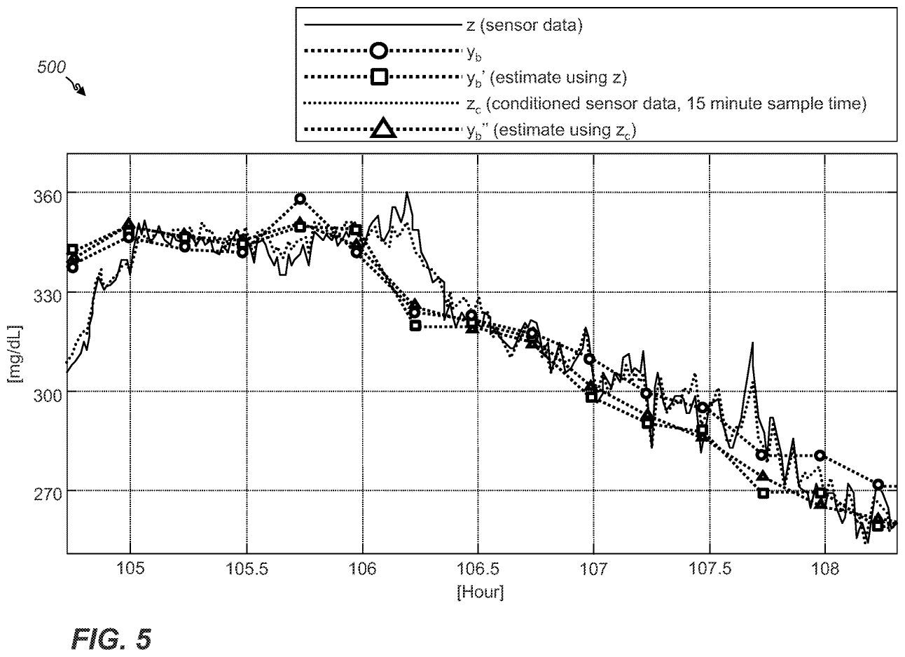

To overcome the attenuation around fast transitions introduced by interpolation filtering methods and the exaggerated fast transitions introduced by extrapolation filtering methods, the methods of the present disclosure combine interpolation based estimates and extrapolation based estimates. As shown in the graph 400 of FIG. 4, when the interpolation-based calculation for curve y_{c} (LS fit based calculation inside the measurement window) and the extrapolation-based calculation for curve yₛ (LS fit based calculation outside the measurement window) are combined, the result is curve yₐ. Note that curve yₐ traces plot z relatively accurately in this example that does not contain significant noise to reject and/or data loss to recover. However, turning now to FIG. 5, the efficacy of this combination in rejecting noisy sensor data is graphically demonstrated.

FIG. 5 is a graph 500 that depicts methods of the present disclosure applied to an example of a noisy sensor data segment. Plot z represents noisy raw sensor data sampled and presented in one minute increments. Curve y_{b} represents reference glucose measurements taken every fifteen minutes, visually connected by dotted lines. When the noisy sensor data of plot z is used for a sensor output calculation that involves a rate of change calculation, the resulting output at fifteen minute intervals is shown as curve y_{b}′. Plot z_{c} represents conditioned sensor data sampled and presented in fifteen minute increments. If the same sensor output calculation uses the conditioning of the present disclosure (i.e., uses plot z_{c} as the input data) instead of plot z, the resulting output at fifteen minute intervals is shown as curve y_{b}″. As can be seen in FIG. 5, using the methods of the present disclosure, there is a significant reduction of the noise in the analyte measurement system output in the presence of noisy raw analyte sensor data input. Further, as illustrated in FIG. 4, the estimation methods of the present disclosure do not attenuate the amplitude of true analyte sensor data excursions and sensor data segments with low noise are not affected.

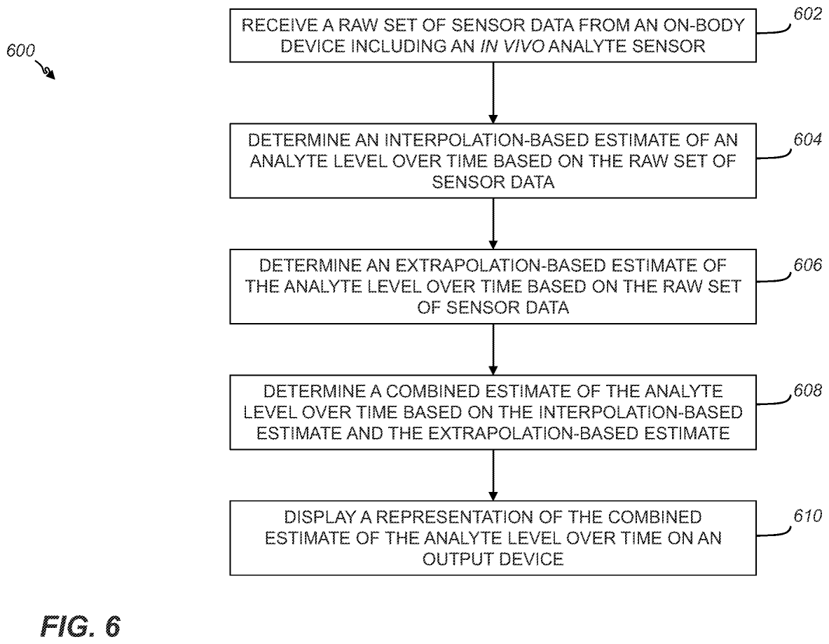

Turning now to FIG. 6, a flow chart 600 depicting example methods of the present disclosure is provided. As indicated above, the methods of the present disclosure can be implemented on a computer or other processing device. In some embodiments, raw sensor data is received from an on-body device that includes an in vivo analyte sensor (602). The raw sensor data may represent data sampled over a period of time during the use of the on-body device. The sample rate may be less than ten or fifteen minutes such that the data collected is sparsely sampled as defined above. In some embodiments, the set of data received may include data collected and stored over an entire wear period.

An interpolation-based estimate of the analyte level over time is determined based on the raw set of sensor data (604). The interpolation-based estimate can be computed based on a least squares fit based calculation of analyte sensor data values within a predefined measurement window. For example, given values z(t0), z(t1), z(t2), up to z(tN), the estimate y_{c}(te) at time te as well as the slope v_{c}(te) at time te based on a least-squares fit of a line can be computed by the following equation:

$$[ \begin{matrix}yc ( te ) \\ vc ( te )\end{matrix} ] = {[ {Φ}^{T} Φ ]}^{- 1} {Φ}^{T} Y$$

where:

$$Y = ⌊ \begin{matrix}z ( \cdot {0}_{)} \\ ⋮ \\ z ( tN )\end{matrix} ⌋ , Φ = [ \begin{matrix}1 & t 0 - te \\ ⋮ & ⋮ \\ 1 & tN - te\end{matrix} ⌉$$

Without loss of generality, suppose only up to three values of z are used to form a window at any time, and that the values are spaced at regular sample interval Ts. Then, for the least-squares estimate at the center:

$$t 1 = te - Ts$$

$$t 2 = te$$

$$t 3 = te + Ts$$

The estimated values y_{c}(te) and v_{c}(te) are then computed as follows:

$$⌊ \begin{matrix}yc ( te ) \\ vc ( te )\end{matrix} ⌋ = {[ {Φ}^{T} Φ ]}^{- 1} {Φ}^{T} Y$$

where:

$$Y = ⌊ \begin{matrix}z ( t 1 ) \\ z ( t 2 ) \\ z ( t 3 )\end{matrix} ⌋ , Φ = [ \begin{matrix}1 & t 1 - te \\ 1 & t 2 - te \\ 1 & t 3 - te\end{matrix} ⌉ = ⌈ \begin{matrix}1 & - Ts \\ 1 & 0 \\ 1 & Ts\end{matrix} ]$$

Likewise, an extrapolation-based estimate of the analyte level over time is determined based on the raw set of sensor data (606). The extrapolation-based estimate can be computed based on a least squares fit based calculation of analyte sensor data values outside or at the left edge of a predefined measurement window. Then, for the least-squares estimate yl(te) and vl(te) at the left of a window of data values z(t2), z(t3), z(t4) with te at t2:

$$t 2 = te$$

$$t 3 = te + Ts$$

$$t 4 = te + 2 Ts$$

The estimated values yl(te) and vl(te) are then computed as follows:

$$[ \begin{matrix}yl ( te ) \\ vl ( te )\end{matrix} ] = {[ {Φ}^{T} Φ ]}^{- 1} {Φ}^{T} Y$$

where:

$$Y = \vert \begin{matrix}z ( t 2 ) \\ z ( t 3 ) \\ z ( t 4 )\end{matrix} \vert , Φ = [ \begin{matrix}1 & t 2 - te \\ 1 & t 3 - te \\ 1 & t 4 - te\end{matrix} \vert = ⌊ \begin{matrix}1 & \\ 1 & Ts \\ 1 & 2 Ts\end{matrix} ⌋$$

Similarly, the extrapolation-based estimate of the analyte level over time can be computed based on a combination of an extrapolation using a least squares fit based calculation of analyte sensor data values from the right side or at the right edge of a predefined measurement window of preceding values and a second extrapolation using a least squares fit based calculation of analyte sensor data values from the right side or at the right edge of a predefined measurement window of succeeding values. Then, for the least-squares estimate yᵣ(te) and vᵣ(te) at the right of a window of data values z(t0), z(t1), z(t2) with te at t2:

$$t 0 = te - 2 Ts$$

$$t 1 = te - Ts$$

$$t 2 = te$$

The estimated values yₗ(te) and vₗ(te) are then computed as follows:

$$[ \begin{matrix}yr ( te ) \\ vr ( te )\end{matrix} ] = {[ {Φ}^{T} Φ ]}^{- 1} {Φ}^{T} Y$$

where:

$$Y = \vert \begin{matrix}z ( t 0 ) \\ z ( t 1 ) \\ z ( t 2 )\end{matrix} \vert , Φ = [ \begin{matrix}1 & t 0 - te \\ 1 & t 1 - te \\ 1 & t 2 - te\end{matrix} \vert = ⌊ \begin{matrix}1 & - 2 Ts \\ 1 & - Ts \\ 1 & \end{matrix} ⌋$$

A combined estimate of the analyte level over time is determined based on the interpolation-based estimate and the extrapolation-based estimate (608). For example, when all calculations yc, yr, and yl are available, an estimate can be calculated by taking the average of the left and right:

$$ys ( te ) = [ yr ( te ) + yl ( te ) ] / 2$$

Which is then combined with the interpolation based estimate to obtain a final estimated value:

$$ya ( te ) = [ ys ( te ) + yc ( te ) ] / 2$$

Alternatively, a final estimate can be obtained by a weighted average of the calculations yc, yr and yl in a more general manner:

$$ya ( te ) = Kc yc ( te ) + Kl yl ( te ) + Kr yr ( te )$$

where the sum of Kc, Kl and Kr equals 1.

In a more general embodiment, when the number of analyte data points z within a predetermined window may vary, the weights applied to each element of the estimate, for example, yc, yl, and yr, can be a function of the number of available data points. The number of data points available can vary due to certain data points having been disqualified by an upstream data integrity check, having been disqualified by an upstream physiological feasibility check, or having been provided with varying time gaps by an upstream process. Conceptually, elements of the estimate such as yc, yl, or yr, whose number of available points are lower than the desired amount, will have a lower weighting factor in order for the less reliable measurement to exert less influence into the final estimate ya.

In some embodiments, yc, yl, and yr are calculated in the same manner as previously described. Instead of fixed weights Kc, Kl, and Kr as previously described, Kc, Kl, and Kr can take on different values as a function of the number of available data points z in their respective windows. Let the number of available data points be denoted Qc, Ql, and Qr. Then, Kc, Kl, and Kr are such that when Qc, Ql, and Qr is equal to the maximum number of points, Kc, Kl, and Kr will take on their largest possible respective values. As Qc, Ql, and Qr approach zero, then Kc, Kl, and Kr will take on their smallest possible respective values, which may or may not be zero. One way to achieve this is to use a smooth function that relates Kc, Kl, and Kr to Qc, Ql, and Qr, respectively. Alternatively, Qc, Ql, and Qr may affect the weights Kc, Kl, and Kr in stepwise thresholds.

A numerical example of the embodiment described, using stepwise thresholds is described as follows. For the calculation of yc, find 3 available data points z as previously described. If the number of valid points is greater than 2 (i.e., Qc>2), set Kc to 5. If the number of valid points is equal to 2 (i.e., Qc=2), set Kc to 2.5. Otherwise, set Kc to 0. This can be achieved by using a function evaluated at the discrete available number of points Qc, or by evaluating Qc against threshold value 2. For the calculation of yl, find 3 available data points z as previously described. If Ql>2, set Kl to 1. If Ql=2, set Kl to 0.4. Otherwise, since there is insufficient number of points, set Kl to 0. For the calculation of yr, find 3 available data points z as previously described. If Qr>2, set Kr to 1. If Qr=2, set Kr to 0.4. Otherwise, set Kr to 0. In addition, if both yl and yr can be calculated, calculate the mean of both values, ym=[yl+yr]/2. A new weight Km is assigned the value 6 if both can be calculated or 0 otherwise. Finally, an estimate that is robust to data loss and can generate results under partially missing data, yf, can be computed by taking the weighted average:

$$yf = [ Kc * yc + Kl * yl + Kr * yr + Km * ym ] / [ Kc + Kl + Kr + Km ] ,$$

when at least one of the weights Kc, Kl, Kr, or Km is nonzero. If all of the weights are zero, there is insufficient data to generate a reliable estimate, and no estimate yf is given.

A representation of the combined estimate of the analyte level over time can then be displayed on an output device operatively coupled to the processor (610). The representation can be, for example, in the form of a graphical plot, a numerical display, or a combination thereof.

In the manner described above, in certain embodiments of the present disclosure, there is provided a method of estimating an analyte level using sparsely sampled analyte sensor data comprising: determining, using a processor, a composite estimate of an analyte level over time based on a combination of an interpolated estimate of the analyte level and an extrapolated estimate of the analyte level, and displaying a representation of the composite estimate of the analyte level over time on an output device.

In certain embodiments, the interpolated estimate of the analyte level and the extrapolated estimate of the analyte level are computed based on a raw set of sensor data.

In certain embodiments, the raw set of sensor data is received from an on-body device including an in vivo analyte sensor.

In certain embodiments, the interpolated estimate of the analyte level over time is computed based on a least squares fit based calculation of analyte sensor data values within a predefined measurement window, and further, wherein the extrapolated estimate of the analyte level over time is computed based on more than one least squares fit based calculation of analyte sensor data values outside or at the edge of the predefined measurement window.

In certain embodiments, the interpolated estimate of the analyte level over time is computed based on a least squares fit based calculation of analyte sensor data values within a predefined measurement window, and further, wherein the extrapolated estimate of the analyte level over time is computed based on a combination of a first extrapolation using a least squares fit based calculation of analyte sensor data values outside or at the edge of a first predefined measurement window and a second extrapolation using a least squares fit based calculation of analyte sensor data values outside or at the edge of a second predefined measurement window.

In certain embodiments, the first predefined measurement window uses analyte sensor data values from a time before a data point of interest and wherein the second predefined measurement window uses analyte sensor data values from a time after the data point of interest.

A computer-implemented method in certain embodiments includes receiving a raw set of sensor data from an on-body device including an in vivo analyte sensor, determining an interpolation-based estimate of an analyte level over time based on the raw set of sensor data, determining an extrapolation-based estimate of the analyte level over time based on the raw set of sensor data, determining a combined estimate of the analyte level over time based on the interpolation-based estimate and the extrapolation-based estimate, and displaying a representation of the combined estimate of the analyte level over time on an output device.

In certain embodiments, the interpolation-based estimate of the analyte level over time based on the raw set of sensor data is computed based on a least squares fit based calculation of analyte sensor data values within a predefined measurement window.

In certain embodiments, the extrapolation-based estimate of the analyte level over time based on the raw set of sensor data is computed based on more than one least squares fit based calculation of analyte sensor data values outside or at the edge of a predefined measurement window.

In certain embodiments, the extrapolation-based estimate of the analyte level over time based on the raw set of sensor data is computed based on a combination of a first extrapolation using a least squares fit based calculation of analyte sensor data values outside or at the edge of a first predefined measurement window and a second extrapolation using a least squares fit based calculation of analyte sensor data values outside or at the edge of a second predefined measurement window.

In certain embodiments, the first predefined measurement window uses analyte sensor data values from a time before a data point of interest and wherein the second predefined measurement window uses analyte sensor data values from a time after the data point of interest.

In certain embodiments, the raw set of sensor data includes data sampled at a rate less than once per ten minutes.

In certain embodiments, the representation of the combined estimate of the analyte level over time includes at least one of a graph and a numeric display.

A system for monitoring analyte concentration in certain embodiments includes a processor, and a memory coupled to the processor, the memory storing processor executable instructions to: receive a raw set of sensor data from an on-body device including an in vivo analyte sensor, determine an interpolation-based estimate of an analyte level over time based on the raw set of sensor data, determine an extrapolation-based estimate of the analyte level over time based on the raw set of sensor data, determine a combined estimate of the analyte level over time based on the interpolation-based estimate and the extrapolation-based estimate, display a representation of the combined estimate of the analyte level over time on an output device operatively coupled to the processor.

In certain embodiments, the instruction to determine the interpolation-based estimate of the analyte level over time based on the raw set of sensor data includes an instruction to determine the interpolation-based estimate based on a least squares fit based calculation of analyte sensor data values within a predefined measurement window.

In certain embodiments, the instruction to determine the extrapolation-based estimate of the analyte level over time based on the raw set of sensor data includes an instruction to determine the extrapolation-based estimate based on more than one least squares fit based calculation of analyte sensor data values outside or at the edge of a predefined measurement window.

In certain embodiments, the instruction to determine the extrapolation-based estimate of the analyte level over time based on the raw set of sensor data includes an instruction to determine the extrapolation-based estimate based on a combination of a first extrapolation using a least squares fit based calculation of analyte sensor data values outside or at the edge of a first predefined measurement window and a second extrapolation using a least squares fit based calculation of analyte sensor data values outside or at the edge of a second predefined measurement window.

In certain embodiments, the first predefined measurement window uses analyte sensor data values from a time before a data point of interest and wherein the second predefined measurement window uses analyte sensor data values from a time after the data point of interest.

In certain embodiments, the raw set of sensor data includes data sampled at a rate less than once per ten minutes.

In certain embodiments, the instruction to display the representation of the combined estimate of the analyte level over time on the output device includes an instruction to display at least one of a graph and a numeric display.

A computer-implemented method in certain embodiments includes receiving a raw set of sensor data from an on-body device including an in vivo analyte sensor, determining an interpolation-based estimate of an analyte level over time based on the raw set of sensor data, determining an extrapolation-based estimate of the analyte level over time based on the raw set of sensor data, determining weights of each estimate based on the number of available sensor data used to compute each estimate, determining a combined estimate of the analyte level over time based on the weighted average of the interpolation-based estimate and the extrapolation-based estimate, displaying a representation of the combined estimate of the analyte level over time on an output device.

Various other modifications and alterations in the structure and method of operation of the embodiments of the present disclosure will be apparent to those skilled in the art without departing from the scope and spirit of the present disclosure. Although the present disclosure has been described in connection with certain embodiments, it should be understood that the present disclosure as claimed should not be unduly limited to such embodiments. It is intended that the following claims define the scope of the present disclosure and that structures and methods within the scope of these claims and their equivalents be covered thereby.

1-20. (canceled)

21. A method of monitoring a glucose concentration using a glucose sensor having a processor configured to be positioned in contact with a fluid under a skin layer of a subject, the method comprising:
receiving a request for a glucose level of the subject;
determining, using the processor of the glucose sensor, a composite estimate of the glucose level of the subject over a period of time based on a combination of an interpolated estimate of the glucose level of the subject that indicates the glucose level within the period of time and an extrapolated estimate of the glucose level of the subject that indicates the glucose level outside the period of time; and
providing, to a display associated with the glucose sensor, the composite estimate of the glucose level of the subject.

22. The method of claim 21, wherein the glucose level over the period of time is sparsely sampled analyte sensor data generated by the glucose sensor.

23. The method of claim 21, wherein the interpolated estimate of the glucose level and the extrapolated estimate of the glucose level are computed based on a raw set of data from the glucose sensor.

24. The method of claim 23, wherein the raw set of data from the glucose sensor includes data sampled at a rate less than once per minute.

25. The method of claim 21, wherein glucose sensor is an in vivo analyte sensor.

26. The method of claim 21, wherein the interpolated estimate of the glucose level is based on a least squares fit based calculation.

27. The method of claim 21, wherein the extrapolated estimate of the glucose level is based on a more than one least squares fit based calculation.

28. The method of claim 21, wherein the extrapolated estimate of the glucose level is based on a combination of a first extrapolation based on a least squares fit based calculation and a second extrapolation based on a least squares fit based calculation.

29. The method of claim 21, wherein the display associated with the glucose sensor is configured to display the extrapolated estimate of the glucose level that includes at least an estimated glucose value outside the period of time.

30. A system for monitoring glucose concentration, the system comprising:
a processor; and
memory coupled to the processor, the memory storing instructions to:
receive a request for a glucose level of a subject; and
determine a composite estimate of a glucose level of the subject over a period of time based on a combination of an interpolated estimate of the glucose level of the subject that indicates the glucose level within the period of time and an extrapolated estimate of the glucose level of the subject that indicates the glucose level outside the period of time, wherein the composite estimate of the glucose level is determined based on data sampled by a glucose sensor that is configured to be positioned in contact with a fluid under a skin layer of a subject.

31. The system of claim 30, wherein the instructions are further configured to cause the system to display a representation of the composite estimate of the analyte glucose level.

32. The system of claim 30, wherein the glucose level over the period of time is sparsely sampled analyte sensor data generated by the glucose sensor.

33. The system of claim 30, wherein the interpolated estimate of the glucose level and the extrapolated estimate of the glucose level are computed based on a raw set of data from the glucose sensor.

34. The system of claim 33, wherein the raw set of data from the glucose sensor includes data sampled at a rate less than once per minute.

35. The system of claim 30, wherein glucose sensor is an in vivo glucose sensor.

36. The system of claim 30, wherein the interpolated estimate of the glucose level is based on a least squares fit based calculation.

37. The system of claim 30, wherein the extrapolated estimate of the glucose level is based on a more than one least squares fit based calculation.

38. The system of claim 30, wherein the instructions are further configured to cause the system to display the extrapolated estimate of the glucose level that includes at least an estimated glucose value outside the period of time.

39. An apparatus for monitoring analyte concentration, the apparatus comprising:
an on-body device including an in vivo glucose sensor that is configured to be positioned in contact with a fluid under a skin layer of a subject, wherein the apparatus is configured to:
receive a request for a glucose level of a subject; and
determine a composite estimate of a glucose level over a period of time based on a combination of an interpolated estimate of the glucose level of the subject that indicates the glucose level within the period of time and an extrapolated estimate of the analyte glucose level of the subject that indicates the glucose level outside the period of time.

40. The apparatus of claim 39, wherein the apparatus is further configured to send instructions to an associated display for displaying the extrapolated estimate of the glucose level that includes at least an estimated glucose value outside the period of time.
